(19) United States
(12) Patent Application Publication
(10) Pub. No.: US 2019/0274534 A1
(43) Pub. Date: Sep. 12, 2019

(54) ENDOSCOPY AND HYDROTHERAPY SPECULUM

(71) Applicant: Deborah Solomon, Phoenix, AZ (US)

(72) Inventor: Deborah Solomon, Phoenix, AZ (US)

(21) Appl. No.: 16/250,300

(22) Filed: Jan. 17, 2019

Related U.S. Application Data
(60) Provisional application No. 62/618,994, filed on Jan. 18, 2018.

(51) Int. Cl. A61B 1/31; A61B 1/015; A61M 3/02

(52) U.S. Cl. CPC A61B 1/31; A61B 1/32; A61M 3/0279; A61B 1/015

(57) ABSTRACT

A rectal speculum that can be used in conjunction with a colon hydrotherapy system to perform bowel cleansing before and/or during an endoscopic procedure which is executed in a completely closed system thereby eliminating exposure to fecal material and contaminated fluids.

### CROSS-REFERENCE TO RELATED APPLICATIONS

This application claims priority to provisional patent application having Ser. No. 62/618,994, filed Jan. 18, 2018, which is herein incorporated by reference in its entirety. This patent application is also related to U.S. patent application Ser. No. 13/176,260, issued as U.S. Pat. No. 9,872,952, which is herein incorporated by reference in its entirety.

### FIELD OF INVENTION

The present invention is directed to a rectal speculum that can be used in conjunction with a colon hydrotherapy system to perform bowel cleansing before or during an endoscopic procedure.

### BACKGROUND OF THE INVENTION

An endoscopy is a non-surgical procedure that is used to examine and/or treat a person's digestive tract using a flexible tube (endoscope) with a light and camera attached to it. In order to obtain a clear view of the digestive tract, it is important for all fecal residue to be removed from the digestive tract. Although patients are provided with instructions on how to lea their digestive tracts prior to endoscopic procedures, an amount of fecal residue may still remain in the digestive tract that obscures viewing and treatment. This may result in the need to reschedule the endoscopic procedure which results in a waste of time and resources.

The present invention provides a device that enables an individual performing an endoscopy to utilize temperature and pressure controlled water to clear fecal residue at any point during the endoscopic procedure in order to provide an unobstructed optical view to examine or treat the digestive tract.

### SUMMARY OF THE INVENTION

The present invention is directed to a rectal speculum that can be used in conjunction with a colon hydrotherapy system to cleanse a patient's bowels before or during an endoscopic procedure. The rectal speculum includes a main tube member with a distal end for insertion into a patient's rectum and a proximal end having a pliable watertight seal with an insertion point for an endoscope or colonoscope. The rectal speculum also includes an outlet tube member for removing fluids from the main tube member where the outlet tube member is attached to a side of the main tube member at an angle less than ninety degrees relative to the proximal end of the main tube member and a diameter of the outlet tube member is as large as a diameter of at least a portion of the main tube member. The rectal speculum also includes an inlet tube member connected to and extending from the outlet tube member for directing fluids into the main tube member via the outlet tube member.

The inlet tube member may include a swivel mechanism to enable a fluid line connected to the inlet tube member to rotate in relation to the outlet tube member. The rectal speculum may also include an obturator removably positioned within the main tube member through the pliable watertight seal at the proximal end of the main tube member. The main tube member may also comprise additional features set forth in related application having Ser. No. 13/176,260, issued as U.S. Pat. No. 9,872,952, including, but not limited to, an inner hard layer and outer soft layer, a measurement gauge visible through the outer soft layer, and a thumb rest extending outward from an outer surface of the main tube member opposite the inlet tube member.

In one exemplary embodiment of the present invention, the rectal speculum for use with colon hydrotherapy in conjunction with endoscopic procedures includes a main tube member having an open distal end for insertion into a patient's rectum, an open proximal end opposite the open distal end, and a pliable watertight seal with an opening therein located near the open proximal end of the main tube member, an outlet tube member attached to a side of the main tube member near the open proximal end of the main tube member, and an inlet tube member connected to and extending from the outlet tube member. The rectal speculum may further include a cap member capable of covering the open proximal end of the main tube member and the cap member may include a lip portion and a protrusion where the protrusion extends from a center of the cap member and is capable of fitting within the opening of the pliable watertight seal of the main tube member.

The rectal speculum may further include an obturator capable of being removably positioned within the main tube member of the rectal speculum for facilitating insertion of the rectal speculum in a patient's rectum. The obturator may include an elongated member having a tapered distal end capable of being inserted through the pliable watertight seal of the main tube member and capable of extending through the open distal end of the main tube member and a proximal end having a handle portion for assistance in inserting the obturator through the pliable watertight seal of the main tube member and removing the obturator from the main tube member. Further, the tapered distal end of the obturator may be made of an inner hard layer and an outer soft layer.

The outlet tube member attached to the side of the main tube member may be attached at an angle less than ninety degrees relative to the open proximal end of the main tube member and may have a diameter as large as at least a portion of the main tube member. The inlet tube member may include a swivel mechanism capable of enabling a fluid line connected to the inlet tube member to rotate in relation to the main tube member.

The main tube member of the rectal speculum may be made of an inner hard layer and an outer soft layer. The main tube member of the rectal speculum may further include an annular depression and annular expansions on opposite sides of the annular depression to form an undulating outer surface of the main tube member. The inlet tube member of the rectal speculum may have a diameter that is less than the diameter of the outlet tube member and less than the diameter of main tube member.

In addition, an end of the inlet tube member that is positioned opposite the end of the outlet tube member that is attached to the side of the main tube member may include one or more ribs for securing the outlet tube member to a waste removal hose. An end of the inlet tube member positioned opposite the end of the inlet tube member that is attached to the outlet tube member may also include one or more rubs for securing the inlet tube member to a hose member for providing fluid to the rectal speculum.

In another exemplary embodiment, the rectal speculum of the present invention for use with colon hydrotherapy in conjunction with endoscopic procedures includes a main tube member having an open distal end for insertion into a patient's rectum, an open proximal end located opposite the open distal end, and a pliable watertight seal with an opening therein positioned within an interior of the main tube member and located near the open proximal end of the main tube member, an outlet tube member attached to a side of the main tube member near the open proximal end of the main tube member, an inlet tube member connected to and extending from the outlet tube member, a cap member capable of covering the open proximal end of the main tube member, and an obturator capable of being removably positioned within the main tube member of the rectal speculum for facilitating insertion of the rectal speculum in the patient's rectum.

The cap member may include a lip portion that is capable of engaging with an outer surface of the main tube member about the open proximal end of the main tube member and a protrusion extending from a center of the cap member such that it is capable of extending into the open proximal end of the main tube member and fitting within the opening of the pliable watertight seal positioned within the interior of the main tube member. Further, the obturator may take the form of an elongated member having a tapered distal end capable of being inserted through the pliable watertight seal positioned within the main tube member and capable of extending through the open distal end of the main tube member and a proximal end having a handle portion for assistance in inserting the obturator through the pliable watertight seal positioned within the main tube member and removing the obturator from the main tube member. In addition, the outlet tube member attached to the side of the main tube member may be attached at an angle less than ninety degrees relative to the open proximal end of the main tube member and may have a diameter as large as at least a portion of the main tube member. Further, the inlet tube member may include a swivel mechanism capable of enabling a fluid line connected to the inlet tube member to rotate in relation to the main tube member.

Finally, like the previously described exemplary embodiment, this second exemplary embodiment may have a main tube member that is made of an inner hard layer and an outer soft layer and the main tube member of the rectal speculum may further include an annular depression and annular expansions on opposite sides of the annular depression to form an undulating outer surface of the main tube member. The inlet tube member of the rectal speculum may also have a diameter that is less than the diameter of the outlet tube member and less than the diameter of main tube member.

The present invention is also directed to a method for performing colon hydrotherapy in conjunction with an endoscopic procedure which includes the steps of 1) providing a rectal speculum having a main tube member with an open distal end for insertion into a patient's rectum, an open proximal end opposite the open distal end, and a pliable watertight seal with an opening therein located near the open proximal end of the main tube member, an outlet tube member attached to a side of the main tube member near the open proximal end of the main tube member, and an inlet tube member connected to and extending from the outlet tube member, 2) inserting the open distal end of the rectal speculum into the patient's rectum, 3) inserting an endoscope through each of the open proximal end, the pliable watertight seal, and the open distal end of the main tube member of the rectal speculum, 4) attaching a waste removal hose to the outlet tube member of the rectal speculum, 5) attaching a fluid hose to the inlet tube member of the rectal speculum, and 6) providing fluid through the inlet tube member and removing waste and fluid through the outlet tube member while performing an endoscopic procedure using the endoscope.

### BRIEF DESCRIPTION OF THE DRAWINGS

The foregoing and other features of the present disclosure will become more fully apparent from the following description, taken in conjunction with the accompanying drawings. Understanding that these drawings depict only some embodiments in accordance with the disclosure and are, therefore, not to be considered limiting of its scope, the disclosure will be described with additional specificity and detail through use of the accompanying drawings.

FIG. 1 is a perspective view of an exemplary embodiment of the endoscopy and hydrotherapy speculum of the present invention shown with a cap member;

FIG. 2 is a side perspective view of the endoscopy and hydrotherapy speculum shown in FIG. 1;

FIG. 3 is a cross-sectional view of the endoscopy and hydrotherapy speculum shown in FIGS. 1-2, and 4 taken along line F-F in FIG. 4;

FIG. 4 is a bottom perspective view of the endoscopy and hydrotherapy speculum shown in FIGS. 1-3;

FIG. 5 is a perspective view of the exemplary embodiment of the endoscopy and hydrotherapy speculum of the present invention shown in FIG. 1 with the cap member removed and an obturator inserted into the speculum;

FIG. 6 is a perspective view of the obturator shown in FIG. 5;

FIG. 7 is a cross-sectional view of the endoscopy and hydrotherapy speculum shown in FIGS. 5 and 8 taken along line G-G in FIG. 8;

FIG. 8 is a bottom perspective view of the endoscopy and hydrotherapy speculum shown in FIG. 5;

FIGS. 9-13 are schematics showing exemplary dimensions of various components of the endoscopy and hydrotherapy speculum of the present invention; and

FIG. 14 shows an exemplary embodiment of the endoscopy and hydrotherapy speculum of the present invention shown with an end cap member attached and an obturator that can be used with the speculum for assisting insertion of the speculum into a patient;

FIG. 15 is the endoscopy and hydrotherapy speculum of the present invention shown in FIG. 14 with the end cap removed and the obturator inserted into the speculum;

FIG. 16 shows the pliable watertight seal located within the main tube member near the open proximal end of the main tube member of the endoscopy and hydrotherapy speculum shown in FIGS. 14 and 15;

FIG. 17 shows the end of an endoscope being inserted into the open proximal end of the main tube member of the endoscopy and hydrotherapy speculum shown in FIGS. 14-16;

FIG. 18 shows the end of the endoscope exiting the open distal end of the main tube member of the endoscopy and hydrotherapy speculum shown in FIGS. 14-17;

FIG. 19 shows the speculum completely connected to the endoscopy and hydrotherapy speculum at the open proximal end of the main tube member of the speculum shown in FIGS. 14-18;

FIGS. 20 and 21 show the endoscope connected to a colon hydrotherapy system that is capable of controlling the flow and temperature of the water through the endoscope;

FIG. 22 shows the endoscopy and hydrotherapy speculum of the present invention connected to a colon hydrotherapy system that is capable of controlling the flow and temperature of the water through the endoscopy and hydrotherapy speculum of the present invention; and

FIG. 23 shows both the endoscope and the endoscopy and hydrotherapy speculum of the present invention connected to a colon hydrotherapy system that is capable of controlling the flow and temperature of the water through both the endoscope and the endoscopy and hydrotherapy speculum of the present invention.

### DETAILED DESCRIPTION OF EXEMPLARY EMBODIMENTS

The endoscopy and hydrotherapy speculum of the present invention is utilized in conjunction with a colon hydrotherapy system and an endoscope during air insufflations, water infusion, water immersion, or a combination thereof, colonoscopy, sigmoidoscopy or other endoscopic G.I. procedures. One example of a colon hydrotherapy system that can be used in conjunction with the endoscopy and hydrotherapy speculum of the present invention is the Hydro San Plus Bowel Care System made by Specialty Health Products, Inc.

The endoscopy and hydrotherapy speculum of the present invention is designed as a multipurpose device. The endoscopy and hydrotherapy speculum of the present invention serves as a specula/speculum when inserted into the rectum and connected via waste and water lines to a colon hydrotherapy systems for pre procedure bowel cleansing. When used by itself in this manner with a colon hydrotherapy system but without an endoscope, the endoscopy and hydrotherapy speculum of the present invention includes a cap member that covers the end of the main tube member not inserted into the patient's rectum so that fluid is provided through the inlet tube member of the speculum and waste is removed through the outlet tube member of the of the speculum. The endoscopy and hydrotherapy speculum is specifically designed to facilitate the removal of residual fecal material due to inadequate bowel cleansing.

The endoscopy and hydrotherapy speculum of the present invention also serves to cleanse the bowel and remove fecal material during an endoscopic procedure to ensure that a physician or medical practitioner is able to maintain a clear view of the internal parts of the body being examined. The endoscopy and hydrotherapy speculum's additional portal provides an insertion point for an endoscope/colonoscope through a pliable watertight seal that is located within an interior of the main tube member of the speculum. Fluid (such as purified water, for example) is provided through the inlet tube member of the speculum and waste is removed through the outlet tube member of the of the speculum. Once inserted into the endoscopy and hydrotherapy speculum of the present invention, the endoscope is unrestricted and unencumbered to perform normally during an air, water or combination G I procedure.

The utilization of the endoscopy and hydrotherapy speculum of the present invention with an endoscope and a colon hydrotherapy system provides the physician or medical practitioner with the ability to utilize temperature and pressure controlled purified water via the endoscope to clear residual fecal residue at any point during the procedure thereby providing an unobstructed cal view. The excess water and fecal residue are removed via the endoscopy and hydrotherapy speculum's outlet tube member waste hose portal, through a colon hydrotherapy system unit, and into the drain/sewer line. This is a completely closed system which eliminates exposure to fecal residue and contaminated water. The endoscopy and hydrotherapy speculum of the present invention allows a clean and sanitary environment for both the patient and the medical staff. The endoscopy and hydrotherapy speculum of the present invention is a unique and unparalleled solution to the growing percentage of inadequate and/or incomplete endoscopic procedures.

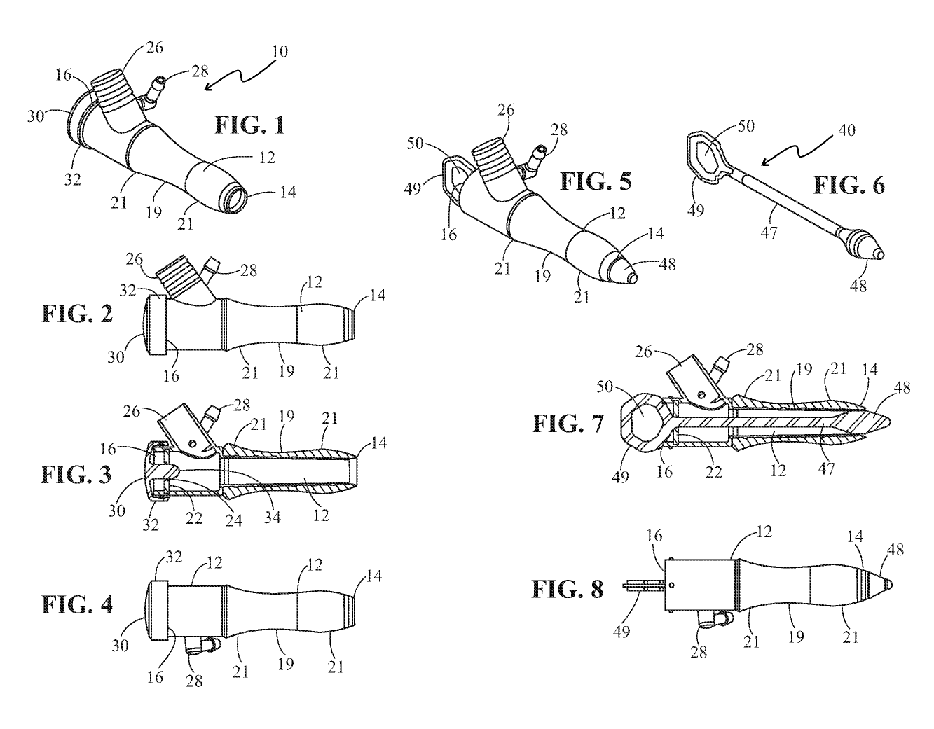

FIG. 1 is a perspective view of an exemplary embodiment of the endoscopy and hydrotherapy rectal speculum 10 of the present invention shown with a cap member for use during pre-procedure bowel cleansing. Rectal speculum 10 includes a main tube member 12 having an open distal end 14 for insertion into a patient's rectum, an open proximal end 16 located opposite the open distal end 14, and a pliable water tight seal 22 (See FIG. 3) an opening 24 contained therein located near the open proximal end 16 of the main tube member 12. The rectal speculum. 12 also includes an outlet tube member 26 attached to a side of the main tube member 12 near the proximal open end 16 of the main tube member 12 such that an interior of outlet tube member 26 is in communication with an interior of main tube member 12. During use, outlet tube member 26 is connected to a waste removal hose for removing waste and fluid which exits the patient's bowels. The rectal speculum 12 also includes an inlet tube member 28 connected to, and extending from, the outlet tube member 26 such that an interior of the inlet tube member 28 is in communication with an interior of the outlet tube member 26. During use, the inlet tube member 28 is connected to a fluid line for directing fluid into the outlet tube member 26, through the main tube member 12, and out the distal end 14 of the main tube member 12 and into a patient's bowels.

In this exemplary embodiment of the invention, the endoscopy and hydrotherapy rectal speculum 10 further includes a cap member 30 which covers the open proximal end 16 of the main tube member 12 so that the rectal speculum 10 of the present invention can be used for pre-procedure bowel cleansing. The pre-procedure bowel cleansing is performed by providing a fluid, such as purified water, to the inlet tube member 28 by connecting a fluid line to the inlet tube member 28 and by evacuating waste and fluid from the patient's bowels through the outlet tube member 26 by connecting a waste removal hose to the outlet tube member 26. The cap member 30 may include a lip 32 which surrounds an outer exterior surface of the main tube member 12 lying adjacent to the open proximal end 16 of the main tube member 12. The cap member 30 may also include a protrusion 34 as shown in FIG. 3.

The main tube member 12 may be configured such that an outer surface of the main tube member 12 includes an annular depression 19 and annular expansions 21 located on opposite sides of the annular depression 19 to create an undulating outer surface of the rectal speculum 10. In addition, the inlet tube member 28 may include a swivel mechanism 29 capable of enabling a fluid line connected to the inlet tube member 28 to rotate in relation to the outlet tube member 26 thereby enabling a user to position the fluid line so that it does not encumber use of the rectal speculum 10. FIG. 2 shows a side perspective view of the endoscopy and hydrotherapy speculum shown in FIG. 1 and FIG. 4 shows a bottom perspective view of the endoscopy and hydrotherapy speculum shown in FIG. 1.

A cross-sectional view of the endoscopy and hydrotherapy speculum 10 of the present invention taken along line F-F of FIG. 4 is shown in FIG. 3. As shown in FIG. 3, the main tube member 12 includes a pliable watertight seal 22 having an opening 24 (also see FIG. 16) located within the main tube member 12 near the proximal end 16 of the main tube member 12. Cap member 30 includes lip 32 which surrounds an outer exterior surface of the main tube member 12 lying adjacent to the open proximal end 16 of the main tube member 12 and also includes a protrusion 34 extending from the center of the cap member 30 such that the protrusion 34 is capable of fitting within the opening 24 of the pliable watertight seal 22 of the main the member 12. As further shown in FIG. 3, main tube member 12 may comprise more than one interior diameter, namely a first larger interior diameter that is in communication with the outlet tube member 26 and a second smaller interior diameter that is the same as the diameter of the open distal end 14 of the main tube member 12.

FIG. 5 is a perspective view of the exemplary embodiment of the endoscopy and hydrotherapy speculum of the present invention shown in FIG. 1 with the cap member 30 removed and an obturator 40 inserted into the speculum. FIG. 6 is a perspective view of the obturator 40 shown in FIG. 5. Obturator 40 is capable of being removably positioned within the main tube member 12 of the rectal speculum 10 for facilitating insertion of the rectal speculum 10 in the patient's rectum. Obturator 40 includes an elongated member 47 having a tapered distal end 48, a proximal handle end 49, and an opening 50 in the proximal handle end 49.

FIG. 8 is a bottom perspective view of the endoscopy and hydrotherapy speculum shown in FIG. 5 and FIG. 7 is a cross-sectional view of the endoscopy and hydrotherapy speculum shown in FIGS. 5 and 8 taken along line G-G in FIG. 8. As shown in FIG. 7, the tapered distal end 48 of the elongated member 47 of the obturator 40 is capable of being inserted through the pliable watertight seal 22 within the main tube member 12 and extending through the open distal end 14 of the main tube member 12. The tapered distal end 48 of the elongated member 47 of the obturator 40 aids in providing a smooth insertion point for more comfortably positioning the rectal speculum 10 within the patient's rectum. The proximal handle end 49 of the elongated member 47 of the obturator 40 aids in inserting the obturator 40 through the pliable watertight seal 22 of the main tube member 12 prior to insertion of the rectal speculum 10 and later aids in removing the obturator 40 from the main tube member 12 after the rectal speculum 10 has been properly positioned within the patient's rectum.

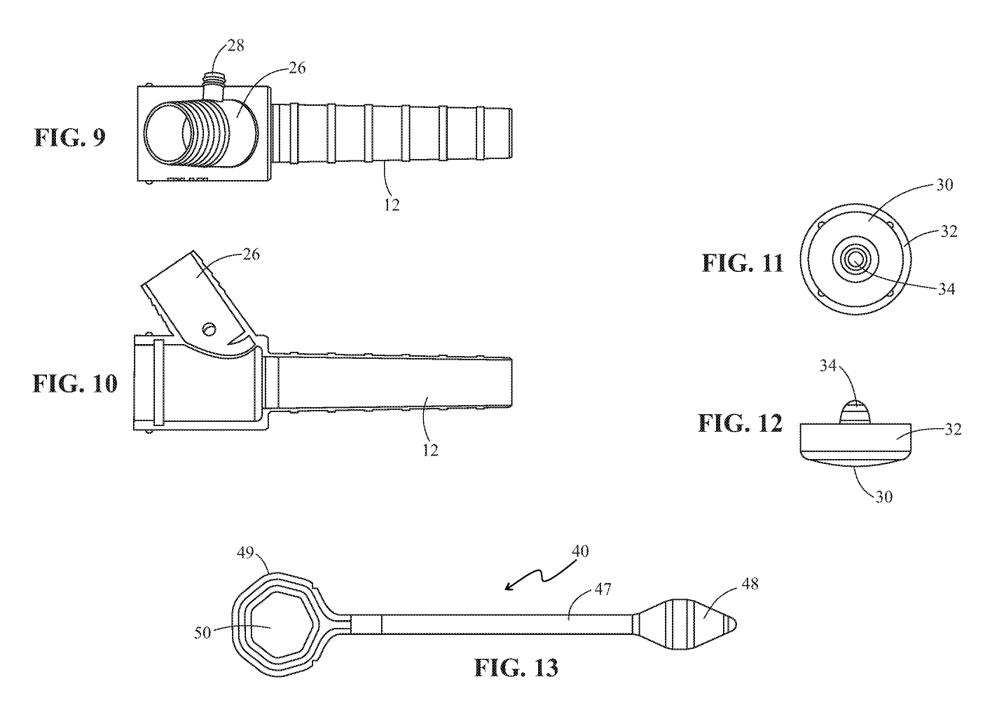

Schematics showing exemplary dimensions of various components of the endoscopy and hydrotherapy speculum 10 of the present invention. FIG. 9 is a side plan view of the rectal speculum 10 and shows an exemplary length measurement of the main tube member 12 of the rectal speculum 10 and an exemplary diameter measurement of the distal end 14 of the main tube member 10. FIG. 10 shows exemplary measurements relating to a cross section of the rectal speculum 10 taken along line B-B of FIG. 9. FIG. 10 shows exemplary length measurements and exemplary inner and outer diameter measurements of the main tube member 12 of the rectal speculum 10 as well as an exemplary inner diameter measurement for outlet tube member 26 and an exemplary angular measurement of the outlet tube member 26 in relation to the main tube member 12. FIG. 11 is a bottom end view of the cap member 30 showing exemplary measurements for the inner and outer diameters of the cap member 30 and FIG. 12 is a side plan view of the cap member 30 showing an exemplary measurement for the length of the protrusion 34 of the cap member 30 which extends beyond the lip 32 of the cap member 30. FIG. 13 is a side plan view of obturator 40 showing exemplary measurements of diameters and angles of the tapered distal end 48 of the elongated member 47 of the obturator 40 and exemplary diameter and length measurements for the elongated member 47 of obturator 40.

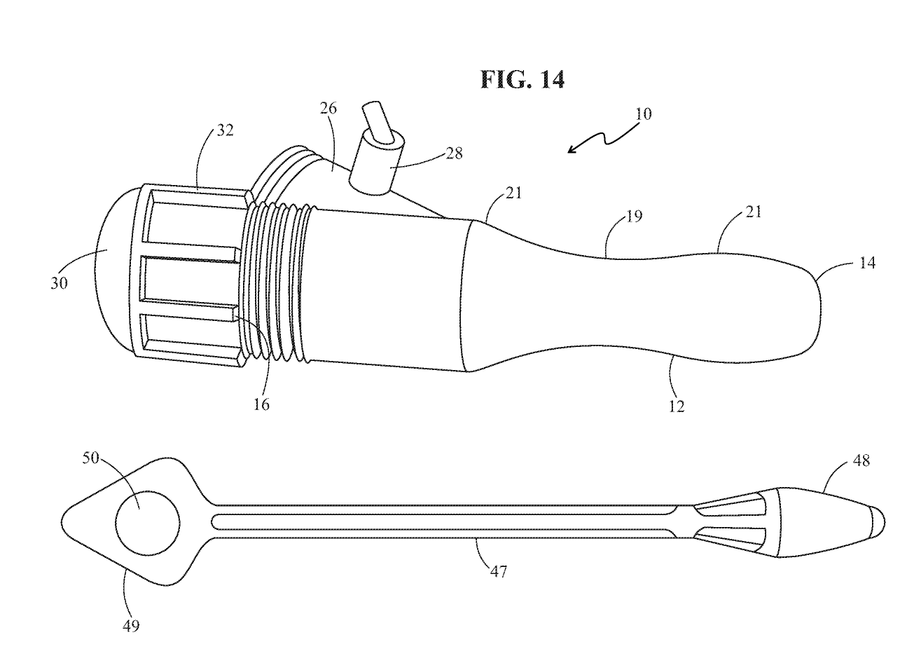

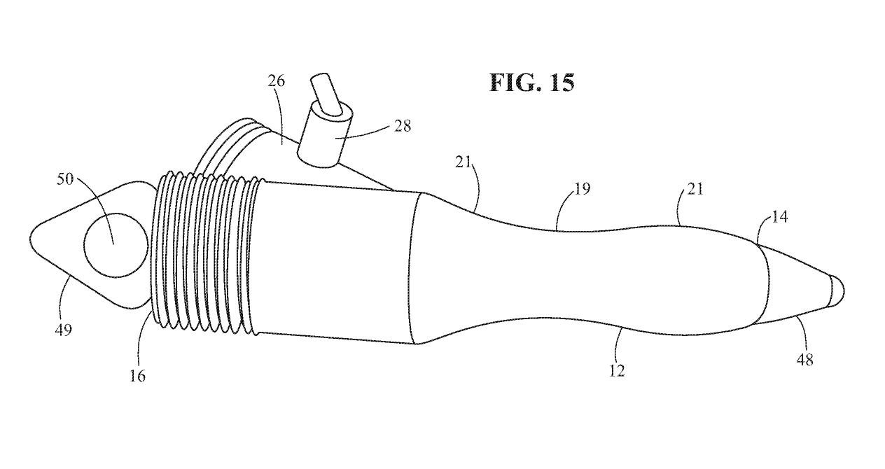

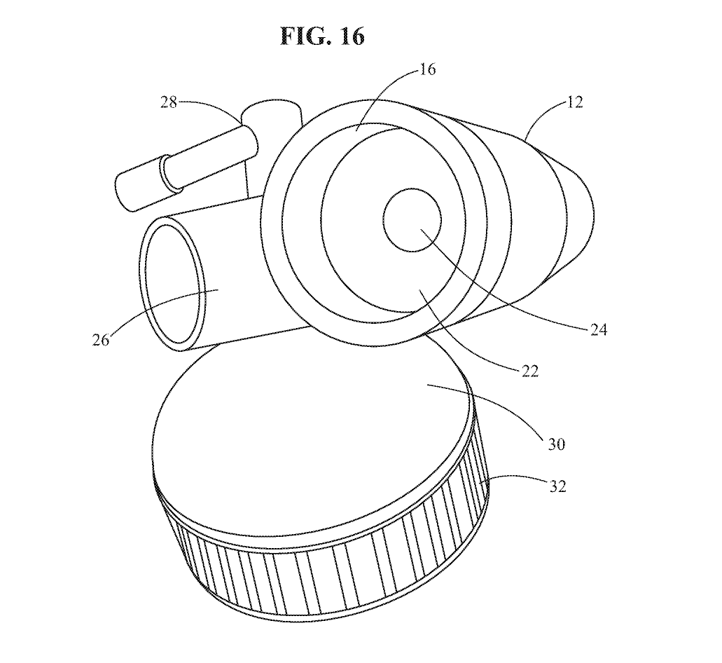

FIG. 14 shows an exemplary embodiment of the endoscopy and hydrotherapy speculum 10 of the present invention shown with an end cap member 30 attached and an obturator 40 that can be used with the speculum 10 for assisting insertion of the speculum 10 into a patient. FIG. 15 is the endoscopy and hydrotherapy speculum 10 of the present invention shown in FIG. 14 with the end cap 30 removed and the obturator 40 inserted into the speculum 10. FIG. 16 shows the pliable watertight seal 22 located within the main tube member 12 near the open proximal end 16 of the main tube member 12 of the endoscopy and hydrotherapy speculum 10 shown in FIGS. 14 and 15. The open proximal end 16 of the main tube member 12 of the speculum 10 is resting on top of removed cap member 30.

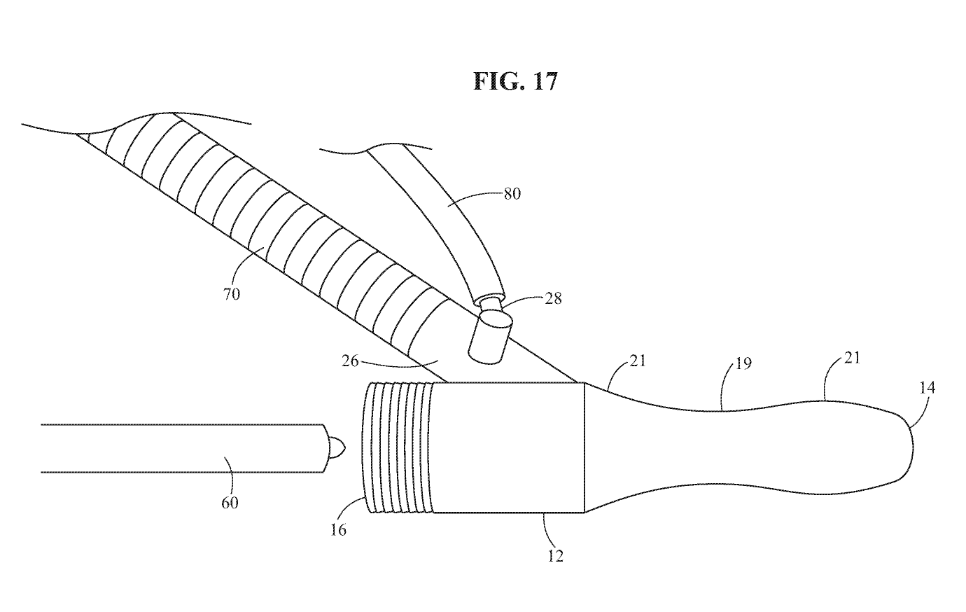

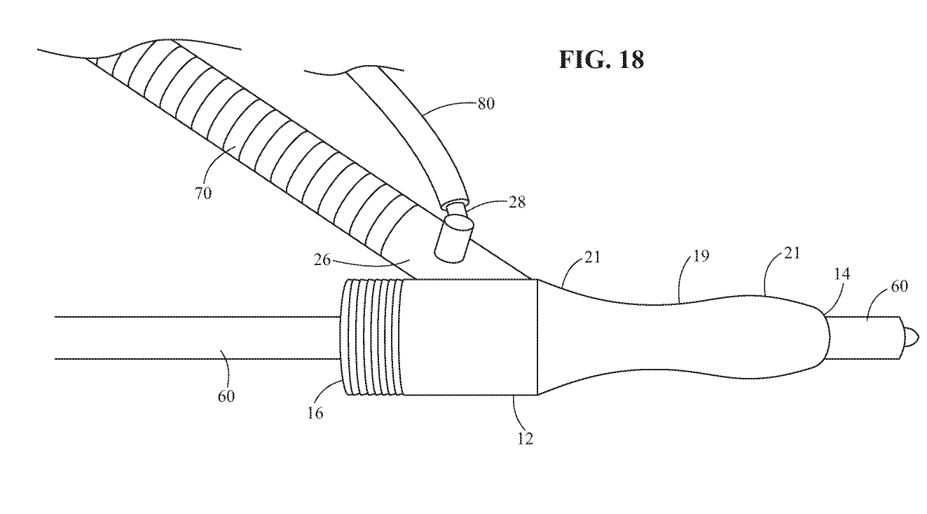

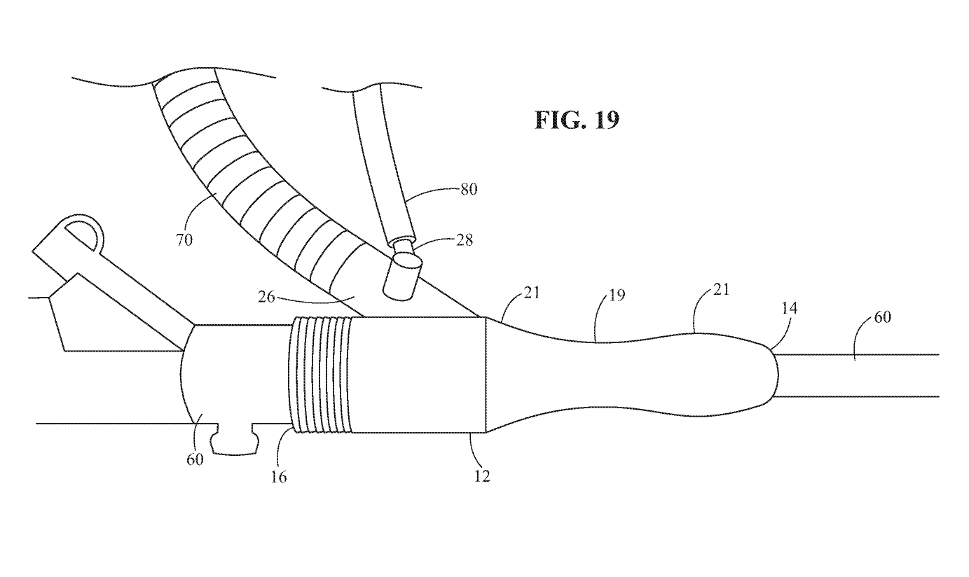

FIG. 17 shows the end of an endoscope 60 being inserted into the open proximal end 16 of the main tube member 12 of the endoscopy and hydrotherapy speculum 10 shown in FIGS. 14-16 with a waste hose 70 connected to the outlet tube member 26 of the speculum 10 and a fluid line/hose 80 connected to the inlet tube member 28 of speculum 10. FIG. 18 shows the end of the endoscope 60 exiting the open distal end 14 of the main tube member 12 of the endoscopy and hydrotherapy speculum 10 shown in FIGS. 14-17 with a waste hose 70 connected to the outlet tube member 26 of the speculum 10 and a fluid line/hose 80 connected to the inlet tube member 28 of speculum 10. FIG. 19 shows the endoscope 60 completely connected to the endoscopy and hydrotherapy speculum 10 at the open proximal end 16 of the main tube member 12 of the speculum 10 shown in FIGS. 14-18 with a waste hose 70 connected to the outlet tube member 26 of the speculum 10 and a fluid line/hose 80 connected to the inlet tube member 28 of speculum 10.

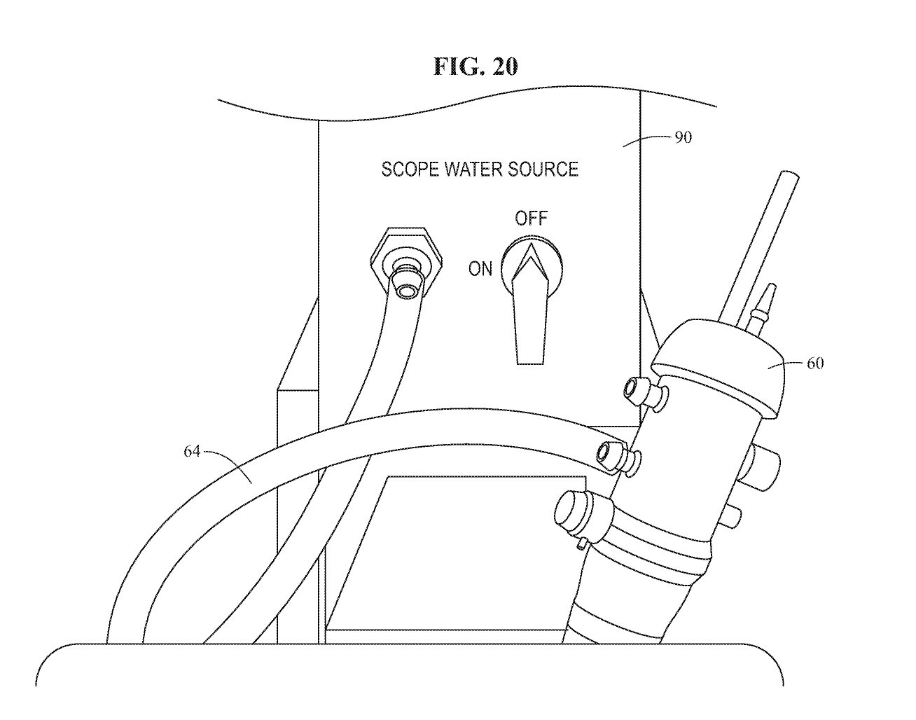

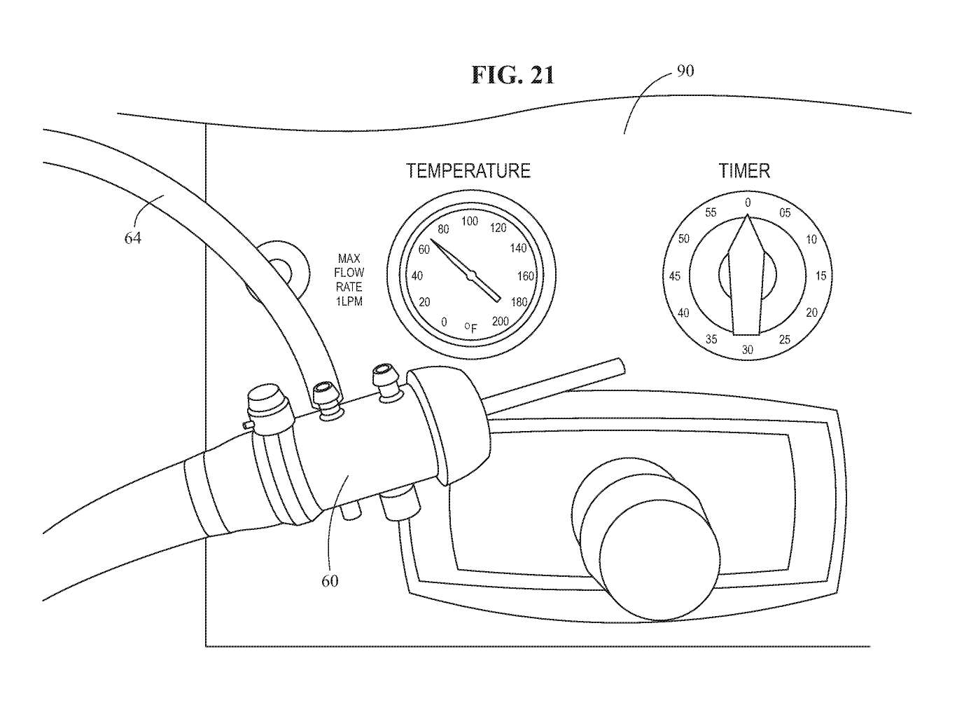

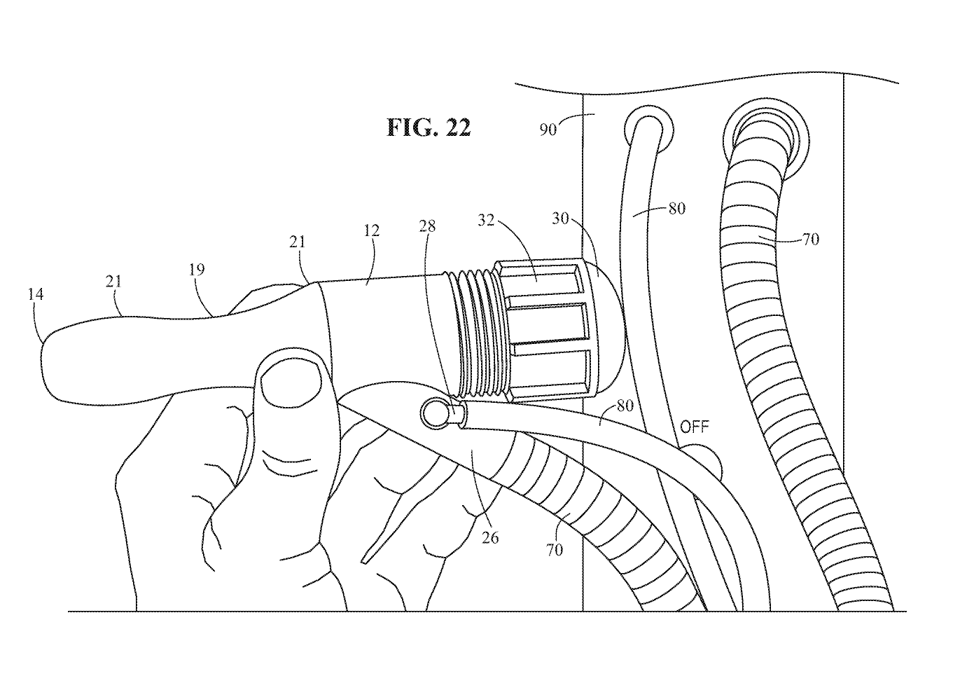

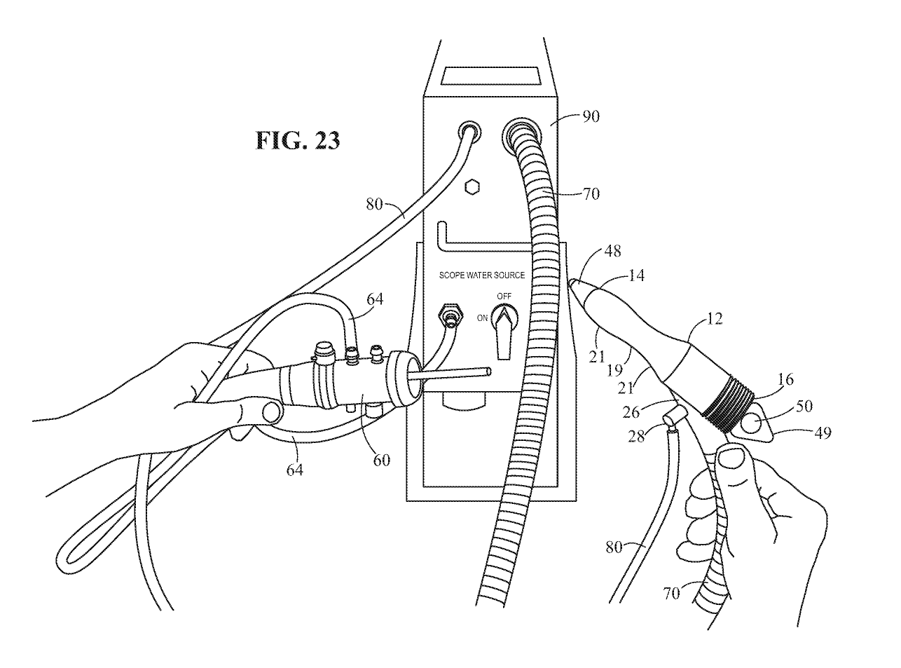

FIGS. 20 and 21 show the endoscope 60 connected to a colon hydrotherapy system 90 via fluid tube 94 that is capable of controlling the flow and temperature of the fluid (e.g. purified water) through the endoscope 60. FIG. 22 shows the endoscopy and hydrotherapy speculum 10 of the present invention being held by an individual's hand and connected to a colon hydrotherapy system 90 that is capable of controlling the flow and temperature of the fluid (e.g. purified water) through the endoscopy and hydrotherapy speculum 10 of the present invention. In this embodiment, cap member 30 covers the open proximal end 16 of the main tube member 12 so that pre-procedure bowel cleansing can be performed prior to removing the cap member 30 and inserting an endoscope through the pliable watertight seal 22 within the main tube member 12 to perform an endoscopic procedure. Waste hose 70 is connected to the outlet tube member 26 of the speculum 10 and to the colon hydrotherapy system 90 and fluid line/hose 80 is connected to the inlet tube member 28 of speculum 10 and to the colon hydrotherapy system 90. Accordingly, when pre-procedure bowel cleansing is performed, it is executed in a completely closed system which eliminates exposure to fecal material and contaminated water. FIG. 23 shows both the endoscope and the endoscopy and hydrotherapy speculum 10 of the present invention connected to a colon hydrotherapy system 90 that is capable of controlling the flow and temperature of the water through both the endoscope 60 and the endoscopy and hydrotherapy speculum 10 of the present invention. An individual's hand is holding an endoscope 60 that is connected to a colon hydrotherapy system 90 via fluid tube 94 and an individual's hand is also holding the endoscopy and hydrotherapy speculum 10 of the present invention with an obturator 40 inserted into the speculum 10 and a waste hose 70 connected to the outlet tube member 26 of the speculum 10 and to the colon hydrotherapy system 90 and fluid line/hose 80 is connected to the inlet tube member 28 of speculum 10 and to the colon hydrotherapy system 90. Once the endoscopy and hydrotherapy speculum 10 of the present invention is inserted into a patient's rectum with the aid of obturator 40, obturator 40 is removed and cap member 30 may be placed over the open proximal end 16 of speculum 10 to perform pre-procedure cleansing of the bowels. After pre-procedure cleansing is performed, the cap member 30 may be removed from the speculum 10 and endoscope 60 can be inserted through pliable watertight seal 22 of the main tube member 12 of the speculum 10 and extended until the endoscope exits the open distal end 14 of the main tube member 10 of the speculum and is secured to the open proximal end 16 of the main tube member 12 of the speculum as shown in FIG. 19. The speculum 10 can be then be used to cleanse the bowel during the endoscopic procedure which utilizes the endoscope 60 that is inserted through the speculum 10. The pliable watertight seal 22 in the main tube member 12 of speculum 10 functions to keep the system sealed so that no fecal residue or contaminated water exits the speculum 10 other than through the outlet tube member 26 of speculum 10. Like the pre-procedure bowel cleansing, the bowel cleansing that takes place during an endoscopic procedure is executed in a completely closed system which eliminates exposure to fecal material and contaminated water by medical staff or anyone else that is located within the vicinity of the procedure.

The present invention is also directed to a colon hydrotherapy system 90 that includes a means for providing a fluid to an endoscope 60, another means for providing a fluid to the endoscopy and hydrotherapy speculum 10 of the present invention, and a means for removing waste which exits the endoscopy and hydrotherapy speculum 10 of the present invention.

The endoscopy and hydrotherapy speculum 10 of the present invention is a rectal speculum and the terms rectal speculum 10, speculum 10, endoscopy and hydrotherapy speculum 10, and endoscopy and hydrotherapy rectal speculum 10 are used interchangeably and are meant to refer to the same thing.

The drawings and description of exemplary embodiments of the invention herein shows various exemplary embodiments of the invention. These exemplary embodiments and modes are described in sufficient detail to enable those skilled in the art to practice the invention and are not intended to limit the scope, applicability, or configuration of the invention in any way. Rather, the following disclosure is intended to teach both the implementation of the exemplary embodiments and modes and any equivalent modes or embodiments that are known or obvious to those reasonably skilled in the art. Additionally, all included examples are non-limiting illustrations of the exemplary embodiments and modes, which similarly avail themselves to any equivalent modes or embodiments that are known or obvious to those reasonably skilled in the art.

Other combinations and/or modifications of structures, arrangements, applications, proportions, elements, materials, or components used in the practice of the instant invention, in addition to those not specifically recited, can be varied or otherwise particularly adapted to specific environments, manufacturing specifications, design parameters, or other operating requirements without departing from the scope of the instant invention and are intended to be included in this disclosure.

Unless specifically noted, it is the Applicant's intent that the words and phrases in the specification and the claims be given the commonly accepted generic meaning or an ordinary and accustomed meaning used by those of ordinary skill in the applicable arts. In the instance where these meanings differ, the words and phrases in the specification and the claims should be given the broadest possible, generic meaning. If any other special meaning is intended for any word or phrase, the specification will clearly state and define the special meaning.

1. A rectal speculum comprising:
a main tube member having an open distal end for insertion into a patient's rectum, an open proximal end opposite the open distal end, and a pliable watertight seal with an opening therein located near the open proximal end of the main tube member;
an outlet tube member attached to a side of the main tube member near the open proximal end of the main tube member; and
an inlet tube member connected to, and extending from, the outlet tube member.

2. The rectal speculum of claim 1 further comprising a cap member capable of covering the open proximal end of the main tube member.

3. The rectal speculum of claim 2 wherein the cap member comprises a lip and a protrusion extending from a center of the cap member wherein the protrusion is capable of fitting within the opening of the pliable watertight seal of the main tube member.

4. The rectal speculum of claim 1 further comprising an obturator capable of being removably positioned within the main tube member of the rectal speculum for facilitating insertion of the rectal speculum in the patient's rectum wherein the obturator comprises an elongated member having a tapered distal end capable of being inserted through the pliable watertight seal of the main tube member and extending through the open distal end of the main tube member and a proximal end having a handle portion for assistance in inserting the obturator through the pliable watertight seal of the main tube member and removing the obturator from the main tube member.

5. The rectal speculum of claim 4 wherein the tapered distal end of the obturator comprises an inner hard layer and an outer soft layer.

6. The rectal speculum of claim 1 wherein the outlet tube member attached to the side of the main tube member is attached at an angle less than ninety degrees relative to the open proximal end of the main tube member and has a diameter as large as a diameter of at least a portion of the main tube member.

7. The rectal speculum of claim 1 wherein the inlet tube member includes a swivel mechanism capable of enabling a fluid line connected to the inlet tube member to rotate in relation to the main tube member.

8. The rectal speculum of claim 1 wherein at least a portion of the main tube member comprises an inner hard layer and an outer soft layer.

9. The rectal speculum of claim 1 wherein the main tube member further comprises an annular depression.

10. The rectal speculum of claim 9 wherein the main tube member further comprises annular expansions on opposite sides of the annular depression.

11. The rectal speculum of claim 1 wherein the inlet tube member has a diameter less than both a diameter of the outlet tube member and a diameter of the main tube member.

12. The rectal speculum of claim 1 wherein an end of the outlet tube member opposite an end of the outlet tube member attached to the side of the main tube member includes one or more ribs for securing the outlet tube member to a waste removal hose.

13. The rectal speculum of claim 1 wherein an end of the inlet tube member opposite an end of the inlet tube member attached to the outlet tube member includes one or more ribs for securing the inlet tube member to a hose member for providing fluid to the rectal speculum.

14. A rectal speculum for use with colon hydrotherapy in conjunction with endoscopic procedures comprising:
a main tube member having an open distal end for insertion into a patient's rectum, an open proximal end opposite the open distal end, and a pliable watertight seal with an opening therein located near the open proximal end of the main tube member;
an outlet tube attached to a side of the main tube member near the open proximal end of the main tube member;
an inlet tube member connected to, and extending from, the outlet tube member;
a cap member capable of covering the open proximal end of the main tube member; and
an obturator capable of being removably positioned within the main tube member of the rectal speculum for facilitating insertion of the rectal speculum in the patient's rectum.

15. The rectal speculum of claim 14 wherein the cap member comprises a lip and a protrusion extending from a center of the cap member wherein the protrusion is capable of fitting within the opening of the pliable watertight seal of the main tube member and the obturator comprises an elongated member having a tapered distal end capable of being inserted through the pliable watertight seal of the main tube member and extending through the open distal end of the main tube member and a proximal end having a handle portion for assistance in inserting the obturator through the pliable watertight seal of the main tube member and removing the obturator from the main tube member.

16. The rectal speculum of claim 14 wherein the outlet tube member attached to the side of the main tube member is attached at an angle less than ninety degrees relative to the open proximal end of the main tube member and has a diameter as large as a diameter of at least a portion of the main tube member.

17. The rectal speculum of claim 14 wherein the inlet tube member includes a swivel mechanism capable of enabling a fluid line connected to the inlet tube member to rotate in relation to the main tube member.

18. The rectal speculum of claim 14 wherein at least a portion of the main tube member comprises an inner hard layer and an outer soft layer.

19. The rectal speculum of claim 14 wherein the inlet tube has a diameter less than both a diameter of the outlet tube member and a diameter of the main tube member.

20. A method for performing colon hydrotherapy in conjunction with an endoscopic procedure comprising the steps of:
providing a rectal speculum having a main tube member with an open distal end for insertion into a patient's rectum, an open proximal end opposite the open distal end, and a pliable watertight seal with an opening therein located near the open proximal end of the main tube member, an outlet tube member attached to a side of the main tube member near the open proximal end of the main tube member, and an inlet tube member connected to and extending from the outlet tube member;
inserting the open distal end of the rectal speculum into the patient's rectum;
inserting an endoscope through each of the open proximal end, pliable watertight seal, and open distal end of the rectal speculum;
attaching a waste removal hose to the outlet tube member of the rectal speculum;
attaching a fluid hose to the inlet tube member of the rectal speculum; and
providing fluid through the inlet tube member and removing waste and fluid through the outlet tube member while performing an endoscopic procedure using the endoscope.
